(19) United States
(12) Patent Application Publication
(10) Pub. No.: US 2016/0279095 A1
(43) Pub. Date: Sep. 29, 2016

(54) USE OF NUTRITIONAL COMPOSITIONS INCLUDING NATURAL VITAMIN E, AND POLYUNSATURATED FATTY ACID

(71) Applicant: ABBOTT LABORATORIES, Abbott Park, IL (US)

(72) Inventors: REMA VAZHAPPILLY, Singapore (SG); CHRON-SI LAI, Blacklick, OH (US); MATTHEW KUCHAN, Westerville, OH (US)

(73) Assignee: Abbott Laboratories, Abbott Park, IL (US)

(21) Appl. No.: 15/036,001

(22) PCT Filed: Nov. 12, 2014
(86) PCT No.: PCT/US2014/065182

§ 371 Date: May 11, 2016

Related U.S. Application Data
(60) Provisional application No. 61/903,120, filed on Nov. 12, 2013.

(51) Int. Cl. A61K 31/355; A61K 31/047; A61K 31/202

(52) U.S. Cl. CPC A61K 31/355; A61K 31/202; A61K 31/047; A23L 1/302; A23L 1/3008; A23L 1/30; A23L 1/296; A23V 2002/00

(57) ABSTRACT

Disclosed herein are nutritional compositions for pregnant or lactating mothers, pre-term and term infants, toddlers and children which include natural vitamin E, a long chain polyunsaturated fatty acid (LC-PUFA) and a carotenoid such as lutein. Also disclosed are methods of administering these compositions for reducing or preventing the susceptibility of the fetal and newborn brain and lungs to hypoxia and the resulting disorders resulting therefrom including abnormal cognitive development, brain in jury and lung injury.

### CROSS-REFERENCE TO RELATED APPLICATIONS

This application claims priority to and any benefit of U.S. Provisional Application No. 61/903,120, filed Nov. 12, 2013, the entire contents of which are incorporated by reference in its entirety.

### FIELD OF THE DISCLOSURE

This disclosure relates to nutritional compositions for pregnant or lactating mothers, pre-term and term infants, toddlers and children which includes natural vitamin E, a long chain polyunsaturated fatty acid (LC-PUFA) and a carotenoid, such as lutein. In addition, this disclosure also relates to the methods of administering these compositions for reducing or preventing the susceptibility of the fetal and newborn brain and lungs to hypoxia and the resulting disorders resulting therefrom including abnormal cognitive development, brain injury and lung injury.

### BACKGROUND OF THE DISCLOSURE

Infant formulas are commonly used today to provide a supplemental or sole source of nutrition early in life to both pre-term and term infants. Similarly, nutritional compositions for pregnant and lactating mothers are also commonly used as supplements for improving fetus and infant health. These formulas typically contain protein, carbohydrate, fat, vitamins, minerals, and other nutrients, and are commercially available as powders, ready-to-feed liquids, and liquid concentrates. Many of these infant formulas provide a quality alternative to human milk as not all infants can receive human milk.

In addition to the nutrients noted above, long chain polyunsaturated fatty acids (LC-PUFAs), including arachidonic acid (ARA) and docosahexaenoic acid (DHA), and tocopherols, including RRR-alpha tocopherol, are present in many infant formulas. ARA and DHA, along with other fatty acids, are generally believed to support brain and vision development in infants, as well as provide other benefits, while tocopherols may provide antioxidant benefits for stabilizing unsaturated lipids in cell membranes against autooxidation and scavenge free radicals produced by lipid peroxidation by the normal activity of oxidative enzymes.

LC-PUFAs included in infant formulas are susceptible to damage by oxidation and degradation. In some cases, a high level of DHA and/or ARA may result in increased generation of lipid peroxides that can degrade RRR-alpha tocopherol before the RRR-alpha tocopherol can be absorbed by the gut.

Additionally, xanthin oxidase (XO) reacts with AMP in the intestine to produce hydrogen peroxide, which may help an infant to prevent bacteria getting through the junction point. However, the newborn infant's anti-oxidation enzymes are not well developed. As a result, the hydrogen peroxide from XO may also oxidize lipids, such as LC-PUFAs, resulting in degradation of the RRR-alpha tocopherol. To combat this unwanted effect, one or more antioxidants can be included in the infant formula to provide some protection from oxidation and degradation of the LC-PUFAs.

There is a continuing need in the art for stable nutritional formulas, including infant formulas as well as nutritional compositions for pregnant and lactating mothers, that can provide a wide range of nutrients to a fetus and/or infant while maintaining stability over an extended period of time.

### SUMMARY OF THE DISCLOSURE

This disclosure is directed to nutritional compositions, and infant formulas in particular, that include the combination of RRR-alpha tocopherol, a LC-PUFA, and lutein or other carotenoid. Also disclosed herein, is the use of these compositions in preventing or reducing the susceptibility of a fetus or infant to neurological and lung disorders, especially those caused by hypoxic injury such as abnormal cognitive development, mental retardation, seizure disorders, cerebral palsy and other brain disorders.

In certain embodiments, the nutritional composition used comprises at least 3 mg/L of RRR-alpha tocopherol and at least 20 mg/L of LC-PUFA. Other embodiments are directed to the use of nutritional compositions comprising at least 3 mg/L of RRR-alpha tocopherol and at least 60 mg/L of LC-PUFA. Still other embodiments are directed to the use of nutritional compositions comprising at least 3 mg/L of RRR-alpha tocopherol and about 72 mg/L to 144 mg/L of LC-PUFA. In all of these embodiments, a carotenoid can optionally be included such as in an amount of 50 to 1150 microgram/L. Lutein, beta-carotene, zeaxanthin, lycopene and combinations thereof, and especially lutein and zeaxanthin, are particularly useful for this purpose. The LC-PUFA that is utilized is at least one of: docosahexaenoic acid, arachidonic acid, docosapentaenoic acid, and gamma linolenic acid.

Additional embodiments are directed to a method for preventing or reducing the susceptibility of a fetus or infant to neurological and lung disorders, especially those caused by hypoxic injury. This method comprises administering to an infant, to a lactating mother, or to a pregnant mother, as the case may be, a nutritional composition comprising at least 7 mg/L of RRR-alpha tocopherol and at least 20 mg/L of LC-PUFA.

Still additional embodiments are directed to a method for preventing or reducing the susceptibility of a fetus or infant to neurological and lung disorders, especially those caused by hypoxic injury, which method comprises administering to an infant, to a lactating mother, or to a pregnant mother, as the case may be, a nutritional composition comprising at least 7 mg/L of RRR-alpha tocopherol and at least 60 mg/L of LC-PUFA.

Still other embodiments are directed to a method for preventing or reducing the susceptibility of a fetus or infant to neurological and lung disorders, especially those caused by hypoxic injury, which method comprises administering to an infant, to a lactating mother, or to a pregnant mother, as the case may be, a nutritional composition comprising at least 3 mg/L of RRR-alpha tocopherol and about 72 mg/L to 144 mg/L of LC-PUFA.

In all of these methods, at least one of docosahexaenoic acid, arachidonic acid, docosapentaenoic acid, and gamma linolenic acid can be utilized as the PC-PUFA, with docosahexaenoic acid or arachidonic acid being especially useful.

In all of these methods, a carotenoid can optionally be included in the composition in the amount of 50 to 1150 micrograms/L. Lutein, beta-carotene, zeaxanthin, lycopene and combinations thereof, and especially lutein and zeaxanthin, are particularly useful for this purpose.

### BRIEF DESCRIPTION OF THE FIGURES

FIG. 1 shows the extent of DHA peroxidation under various conditions described in Example 1.

### DETAILED DESCRIPTION

Docosahexaenoic acid (DHA) peroxidation generates malondialdehyde (MDA), which leads to impaired neuronal function.

In the patent and non-patent literature, Vitamin E has been shown to reduce lipid peroxidation in cigarette smokers. In addition, lutein's neuroprotective effect on the retina has been well demonstrated by several pre-clinical studies.

In addition, it has already been suggested in W02013/138157A1 that nutritional formulas generally, and infant formulas specifically, including a combination of RRR-alpha tocopherol and LC-PUFAs may enhance brain development and improve cognitive performance in an individual, and specifically in an infant. In addition, it has also been suggested in U.S. 2007/0098849 that nutritional formulas generally, and infant formulas specifically, including the combination of Lutein, Vitamin E and LC-PUFAs may enhance cognitive development through visual learning.

However the effect of Lutein and Natural Vitamin E in combination in protecting the brain or neurons from lipid (DHA) peroxidation, and therefore in reducing the susceptibility of pre-term/low birth weight infants, as well as full term infants, to hypoxic injury as well as abnormal cognitive development and brain disorders, has not been suggested in the prior art.

In accordance with this disclosure, it has been found that the susceptibility of the fetal and newborn brain and lungs to hypoxic injury, as well as disorders resulting therefrom including abnormal cognitive development, brain injury and lung injury, can be reduced or prevented by administering the nutritional compositions of this disclosure to the newborn and/or to the mother of the newborn while lactating or even before birth.

These and other elements or features of the various embodiments are described in detail hereafter.

The terms “retort” and “retort sterilized” are used interchangeably herein, and unless otherwise specified, refer to the common practice of filling a container, most typically a metal can or other similar package, with a nutritional liquid, such as a liquid infant formula, and then subjecting the liquid-filled package to the necessary heat sterilization step, to form a retort sterilized nutritional liquid product.

The terms “aseptic” and “aseptic sterilized” are used interchangeably herein, and unless otherwise specified, refer to the manufacture of a packaged product without reliance upon the above-described retort packaging step, wherein the nutritional liquid and package are sterilized separately prior to filling, and then are combined under sterilized or aseptic processing conditions to form a sterilized, aseptically packaged, nutritional liquid product.

The terms “nutritional composition,” “nutritional product,” and “nutritional formula” as used herein, unless otherwise specified, are used interchangeably to refer to nutritional liquids and nutritional powders that comprise at least one of protein, fat, and carbohydrate and are suitable for oral administration to a human. The nutritional composition may further comprise vitamins, minerals, and other ingredients and represent a sole, primary, or supplemental source of nutrition. Nutritional compositions include infant formulas.

The term “nutritional liquid,” as used herein, unless otherwise specified, refers to nutritional products in ready-to-drink liquid form, concentrated form, and nutritional liquids made by reconstituting the nutritional powders described herein prior to use.

The term “nutritional powder,” as used herein, unless otherwise specified, refers to nutritional products in flowable or scoopable form that can be reconstituted with water or another aqueous liquid prior to consumption and includes both spray dried and drymixed/dryblended powders.

The terms “fat,” “lipid” and “oil” as used herein, unless otherwise specified, are used interchangeably to refer to lipid materials derived or processed from plants or animals. These terms also include synthetic lipid materials so long as such synthetic materials are suitable for oral administration to humans.

The term “cognitive performance” as used herein, unless otherwise specified, refers to the learning, thinking, and memory functions (i.e., memory acquisition, memory retention and memory recall) of the brain. Accordingly, the term “improving cognitive performance” as used herein, unless otherwise specified, refers to improving the learning, thinking, and/or memory (memory acquisition, memory retention and memory recall) functions of an infant.

All percentages, parts and ratios as used herein, are by weight of the total product, unless otherwise specified. All such weights as they pertain to listed ingredients are based on the active level and, therefore, do not include solvents or by-products that may be included in commercially available materials, unless otherwise specified.

All references to singular characteristics or limitations of the present disclosure shall include the corresponding plural characteristic or limitation, and vice versa, unless otherwise specified or clearly implied to the contrary by the context in which the reference is made. As used herein, the term “about” for example when used in the context of “ about X” where X is a numerical value, should be understood to also include the exact value of X, as well as variations thereof, for example +/−10%, 5%, 1%, 0.5% or 0.1% of the specified amount X.

All combinations of method or process steps as used herein can be performed in any order, unless otherwise specified or clearly implied to the contrary by the context in which the referenced combination is made.

The various embodiments of the nutritional compositions of the present disclosure may also be substantially free of any ingredient or feature described herein, provided that the remaining formula still contains all of the required ingredients or features as described herein. In this context, and unless otherwise specified, the term “substantially free” means that the selected composition contains less than a functional amount of the optional ingredient, typically less than 1%, including less than 0.5%, including less than 0.1%, and also including zero percent, by weight of such optional or selected essential ingredient.

The nutritional compositions may comprise, consist of, or consist essentially of the elements of the products as described herein, as well as any additional or optional element described herein or otherwise useful in nutritional product applications. For example, as used herein, the term “comprising” is intended to mean that a composition or method includes the recited features, but that other features are not excluded and the term “consisting of” is intended to mean that a composition or method includes only the recited features.

### Product Form

The nutritional compositions of this disclosure may be formulated and administered in any known or otherwise suitable oral product form. Any solid, semi-solid, liquid, semi-liquid, or powder form, including combinations or variations thereof, are suitable for use herein, provided that such forms allow for safe and effective oral delivery to the individual of the essential ingredients as also defined herein.

Specific non-limiting examples of product forms suitable for use with products and methods disclosed herein include, for example, liquid and powder pre-term infant formulas, liquid and powder term infant formulas, liquid and powder toddler formulas, and liquid and powder elemental and semi-elemental formulas. Adult nutritional formulas are also within the scope of the present disclosure.

The nutritional compositions of the present disclosure are preferably formulated as dietary product forms, which are defined herein as those embodiments comprising the ingredients of the present disclosure in a product form that also contains at least one of fat, protein, and carbohydrate. The compositions may be formulated with sufficient kinds and amounts of nutrients to provide a sole, primary, or supplemental source of nutrition, or to provide a specialized nutritional product such as for use in infants afflicted with specific diseases or conditions or with a targeted nutritional benefit.

### Nutritional Liquids

Nutritional liquids include both concentrated and ready-to-feed nutritional liquids. These nutritional liquids are most typically formulated as suspensions, emulsions or clear or substantially clear liquids.

Nutritional emulsions suitable for use may be aqueous emulsions comprising proteins, fats, and carbohydrates. These emulsions are generally flowable or drinkable liquids at from about 1° C. to about 25° C. and are typically in the form of oil-in-water, water-in-oil, or complex aqueous emulsions, although such emulsions are most typically in the form of oil-in-water emulsions having a continuous aqueous phase and a discontinuous oil phase.

The nutritional liquids may be and typically are shelf stable. The nutritional liquids typically contain up to 95% by weight of water, including from about 50% to about 95%, also including from about 60% to about 90%, and also including from about 70% to about 85%, of water by weight of the nutritional liquid. The nutritional liquids may have a variety of product densities, but most typically have a density greater than 1.03 g/mL, including greater than 1.04 g/mL, including greater than 1.055 g/mL, including from about 1.06 g/mL to about 1.12 g/mL, and also including from about 1.085 g/mL to about 1.10 g/mL.

The nutritional liquid may have a pH ranging from about 3.5 to about 8, but are most advantageously in a range of from about 4.5 to about 7.5, including from about 5.5 to about 7.3, including from about 6.2 to about 7.2.

Although the serving size for the nutritional liquid can vary depending upon a number of variables, a typical serving size is generally at least 2 mL, or even at least 5 mL, or even at least 10 mL, or even at least 25 mL, including ranges from 2 mL to about 300 mL, including from about 100 mL to about 300 mL, from about 4 mL to about 250 mL, from about 150 mL to about 250 mL, from about 10 mL to about 240 mL, and from about 190 mL to about 240 mL.

### Nutritional Powders

The nutritional powders are in the form of flowable or substantially flowable particulate compositions, or at least particulate compositions. Particularly suitable nutritional powder forms include spray dried, agglomerated or dryblended powder compositions, or combinations thereof, or powders prepared by other suitable methods. The compositions can easily be scooped and measured with a spoon or similar other device, wherein the compositions can easily be reconstituted with a suitable aqueous liquid, typically water, to form a nutritional liquid, such as an infant formula, for immediate oral or enteral use. In this context, “immediate” use generally means within about 48 hours, most typically within about 24 hours, preferably right after or within 20 minutes of reconstitution. It should be understood that when amounts of certain ingredients are provided in terms of milligrams/L or micrograms/L that these amounts refer to the concentration of the ingredient in a nutritional powder that has been reconstituted such as to be suitable for consumption.

### RRR-Alpha Tocopherol

The nutritional compositions of the present disclosure include RRR-alpha tocopherol. As used herein, the term “RRR-alpha tocopherol” refers to both exogenous sources and inherent sources of RRR-alpha tocopherol and RRR-alpha tocopherol acetate that are present in a nutritional composition, including an infant formula. Inherent sources include RRR-alpha tocopherol that is inherently present in components that are present in a nutritional composition and may include for example, various oils and fats. Exogenous sources of RRR-alpha tocopherol include RRR-alpha tocopherol that is added to the nutritional composition not as part of another component.

It has been discovered that brain accretion of RRR-alpha tocopherol enhances the central nervous system maturation and cognition; that is, the presence of RRR-alpha tocopherol in the brain of a human infant enhances the maturation of the infant's central nervous system and cognitive development. The presence of elevated levels of RRR-alpha tocopherol in the brain may increase the production of cholesterol in the brain, which leads to increased neuron myelination. Also, the brain accretion of RRR-alpha tocopherol stimulates the production of glutamate in the brain, which can result in neuron elongation and branching, which can lead to the establishment of gap junctions between neurons. This gap communication can significantly increase the communication speed between neurons and allow the brain to process more data in a shorter time.

Tocopherols, generically referred to as vitamin E, are available in four forms, alpha, beta, gamma, and delta, which differ in the number and position of the methyl groups on the chroman ring (see Table 1). Further, tocopherols can exist in a number of stereoisomeric forms depending on the chirality of the phytyl tail. Of the alpha tocopherols, RRR-alpha tocopherol (also referred to as “natural vitamin E”) has the greatest biological activity and is reported to be the dominant form of the alpha tocopherol in the brain. RRR-alpha tocopherol is a single stereoisomer whereas synthetic vitamin E (all-rac-alpha tocopherol or tocopherol acetate) is an equimolar mixture of eight isomers, only one of which is RRR-alpha tocopherol. The fact that the dominant form of alpha tocopherol is RRR alpha tocopherol (based on animal studies) strongly suggests that the other seven chiral isomers must be absorbed at a lower rate by the brain or oxidized at a faster rate. Cholesterol is a major component of myelin, and it is likely that stimulated cholesterol synthesis may stimulate newborn infant neuron myelination. Glutamate has been shown to stimulate neurite outgrowth and branching; neurite outgrowth and branching allows neuron cells to establish gap junctions with multiple neurons; the discovery that RRR alpha tocopherol correlates with glutamate and cholesterol suggests that RRR alpha tocopherol plays a key role in newborn infant central nervous system maturation.

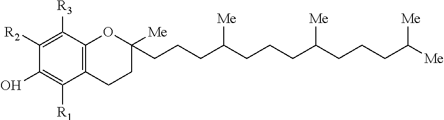

TABLE 1

| Compound | R₁ | R₂ | R₃ |
|---|---|---|---|
| alpha-tocopherol | Me | Me | Me |
| beta-tocopherol | Me | H | Me |
| gamma-tocopherol | H | Me | Me |
| delta-tocopherol | H | H | Me |

The RRR-alpha tocopherol is present in the nutritional compositions in concentrations of at least 3 mg/L, including at least 5 mg/L, including at least 7 mg/L, including at least 10 mg/L, including at least 15 mg/L, including at least 18 mg/L, including at least 20 mg/L, including from at least 7 mg/L to about 100 mg/L, including from at least 7 mg/L to about 50 mg/L, and including from about 20 mg/L to about 40 mg/L, and especially about 25 to 35 mg/L of the nutritional composition. The total amounts of RRR-alpha tocopherol include both exogenous and inherent amounts of RRR-alpha tocopherol, as noted above.

The amount of RRR-alpha-tocopherol that is provided according to the methods and uses of the nutritional compositions provided herein can also be expressed as a total amount of RRR-alpha-tocopherol provided on a per day (/day) basis from consumption of the nutritional composition. According to the methods and uses provided herein, it is generally desirable that the nutritional composition comprises RRR-alpha tocopherol in an amount sufficient to provide a total amount of RRR-alpha tocopherol of at least 3 mg/day, including at least 5 mg/day, including at least 10 mg/day, including at least 20 mg/day, including at least 40 mg/day, including at least 80 mg/day, including at least 100 mg/day, including from at least 200 mg/day to about 300 mg/day, including from at least 500 mg/day. The total amounts of RRR-alpha tocopherol include both exogenous and inherent amounts of RRR-alpha tocopherol, as noted above.

In some embodiments, the nutritional compositions may include another additional tocopherol, particularly gamma-tocopherol, in addition to the RRR-alpha tocopherol. Gamma tocopherol has been used in food applications as an antioxidant, thereby preventing deterioration of foods and beverages resulting from oxidation of susceptible components such as some fats.

Long Chain Polyunsaturated Fatty Acids (LC-PUFAs)

The nutritional compositions of the present disclosure include one or more LC-PUFAs in addition to the RRR-alpha tocopherol. LC-PUFAs are included in the nutritional compositions to provide nutritional support and benefits, as well as to support brain development in individuals, and specifically in infants. Particularly suitable for this purpose are docosahexaenoic acid (DHA), arachidonic acid (ARA), docosapentaenoic acid (DPA), gamma linolenic acid (GLA), and combinations thereof. DHA is especially suitable.

DHA is an n-3 LC-PUFA and is abundant in the brain and retina, accounting for 40% of the LC-PUFAs in the brain and 60% of the LC-PUFAs in the retina. ARA is an n-6 LC-PUFA that is present in the phospholipids, especially phosphatidylethanolamine, phosphatidylcholine, and phosphatidylinositides, of membranes of the body's cells, and is abundant in the brain, muscles, and liver.

The LC-PUFAs may be provided as free fatty acids, in triglyceride form, in diglyceride form, in monoglyceride form, in phospholipid form, or as a mixture of one or more of the above, preferably in triglyceride form. In certain embodiments, when the LC-PUFA is DHA, it is provided in a phospholipid form (i.e., PS-DHA) or a triglyceride form.

In some embodiments, the nutritional compositions include these LC-PUFAs in a concentration of at least 20 mg/L, including at least 40 mg/L, including at least 60 mg/L. Concentrations of LC-PUFAs of about 60-150 mg/L, including about 72-144 mg/L, about 75-130 mg/L, about 80-110 mg/L, and even about 85-100 mg/L, are more interesting. In some embodiments, the nutritional compositions include DHA, ARA, or both in a total concentration of at least 20 mg/L, including at least 40 mg/L, including at least 60 mg/L. Concentrations of DHA, ARA, or both in a total of about 60-150 mg/L, including about 72-144 mg/L, about 75-130 mg/L, about 80-110 mg/L, and even about 85-100 mg/L, are more interesting. In some embodiments, the nutritional compositions utilize DHA as the PC-PUFA in a concentration of at least 20 mg/L, including at least 40 mg/L, including at least 60 mg/L. Concentrations of DHA, of about 60-150 mg/L, including about 72-144 mg/L, about 75-130 mg/L, about 80-110 mg/L, and even about 85-100 mg/L, are more interesting.

The amount of LC-PUFA that is provided according to the methods and uses of the nutritional compositions provided herein can also be expressed as a total amount of LC-PUFA provided on a per day (/day) basis from consumption of the nutritional composition. According to the methods and uses provided herein, it is generally desirable that the nutritional composition comprises one or more LC-PUFAs in an amount sufficient to provide a total amount of at least 5 mg/day, including at least 10 mg/day, including at least 15 mg/day. According to the methods and uses provided herein, amounts of LC-PUFAs in the nutritional compositions sufficient to provide a total amount of LC-PUFAs of about 5-1200 mg/day, including about 10-200 mg/day, about 20-300 mg/day, about 30-500 mg/day, and even about 50-1200 mg/day, are more interesting.

In some embodiments, the nutritional compositions include combinations of RRR-alpha tocopherol, DHA, ARA or both such that the weight ratio of DHA, ARA or both to RRR-alpha tocopherol ranges from about 1:1 to 15:1, more typically about 1.5:1 to 12:1,or even about 1.8:1 to 10:1. When the RRR-alpha tocopherol is used alone, i.e., without a carotenoid also being present as further discussed below, the weight ratio of DHA, ARA or both to RRR-alpha tocopherol normally ranges from about 1:1 to 5:1, more typically about 1.5:1 to 3:1, or even about 1.8:1 to 2.5:1 to achieve the best resistance to peroxidation of the LC-PUFA. However, when a carotenoid is also present as further discussed below, the weight ratio of DHA, ARA or both to RRR-alpha tocopherol normally ranges from about 3:1 to 15:1, more typically about 4:1 to 10:1, or even about 5:1 to 8:1 to achieve the best resistance to peroxidation of the LC-PUFA.

In some embodiments, it may be desirable to supply the LC-PUFA in a microencapsulated form so that it becomes bioavailable only after some predetermined period of time after it is taken up by the infant or fetus being treated. For example, it may be desirable to delay the uptake of the LC-PUFA for at least 2, at least 4, at least 6, at least 8 or at least 10 or more hours after a nutritional composition made in accordance with this disclosure is consumed.

### Carotenoids

The nutritional compositions additionally include carotenoids to reduce or prevent the susceptibility of the fetal and newborn brain and lungs to hypoxia and the resulting disorders resulting therefrom including abnormal cognitive development, brain injury and lung injury. In exemplary embodiments, the nutritional compositions include lutein, beta-carotene, zeaxanthin, lycopene, and combinations thereof. In some embodiments, the nutritional composition includes one or more of lutein and zeaxanthin. Lutein is preferred.

It is generally desirable that the nutritional composition comprises at least one of lutein, lycopene, zeaxanthin, beta-carotene to provide a total amount of carotenoid of from about 50 micrograms/L to about 1150 micrograms/L. More particularly, the nutritional compositions comprise one or more of these carotenoids in a total amount of from 50 micrograms/L to 1150 micrograms/L, including from 100 micrograms/L to 1000 micrograms/L, including from 1 micrograms/L to 300 micrograms/L, and also including from 300 micrograms/L to 1150 micrograms/L. It is also generally desirable that the nutritional compositions comprise lutein in an amount from 50 micrograms/L to 1150 micrograms/L, including from 100 micrograms/L to 1000 micrograms/L, including from 1 micrograms/L to 300 micrograms/L, and also including from 300 micrograms/L to 1150 micrograms/L. In certain embodiments, the nutritional compositions comprise one or more of these carotenoids in a total amount of from 1 microgram/L to 1200 micrograms/L, including from 50 micrograms/L to 1000 micrograms/L, including from 100 micrograms/L to 750 micrograms/L, including from 200 micrograms/L to 500 micrograms/L, including from 250 micrograms/L to 350 micrograms/L, and also including about 300 micrograms/L. In certain embodiments, the nutritional compositions comprise lutein in a total amount of from 1 microgram/L to 1200 micrograms/L, including from 50 micrograms/L to 1000 micrograms/L, including from 100 micrograms/L to 750 micrograms/L, including from 200 micrograms/L to 500 micrograms/L, including from 250 micrograms/L to 350 micrograms/L, and also including about 300 micrograms/L.

The amount of the at least one carotenoid that is provided according to the methods and uses of the nutritional compositions provided herein can also be expressed as a total amount of carotenoid provided on a per day (/day) basis from consumption of the nutritional composition. According to the methods and uses provided herein, it is generally desirable that the nutritional composition comprises at least one of lutein, lycopene, zeaxanthin, beta-carotene in an amount sufficient to provide a total amount of carotenoid of from about 50 micrograms/day to about 1200 micrograms/day. In other words, the person consuming the nutritional composition is provided with a total amount of carotenoid of from about 500 micrograms/day to about 1200 micrograms/day. More particularly, the amount of carotenoid is from 50 micrograms/day to 1200 micrograms/day, including from 100 micrograms/day to 1000 micrograms/day, including from 1 micrograms/day to 300 micrograms/day, and also including from 300 micrograms/day to 1200 micrograms/day. According to the methods and uses provided herein, it is also generally desirable that the nutritional compositions comprise lutein in an amount sufficient to provide from 50 micrograms/day to 1200 micrograms/day, including from 100 micrograms/day to 1000 micrograms/day, including from 1 micrograms/day to 300 micrograms/day, and also including from 300 micrograms/day to 1150 micrograms/day to the person consuming the nutritional composition. According to the methods and uses provided herein, in certain embodiments, the nutritional compositions comprise one or more of these carotenoids in an amount sufficient to provide the person consuming the nutritional compositions with a total amount of carotenoid of from 1 microgram/day to 1200 micrograms/day, including from 50 micrograms/day to 1000 micrograms/day, including from 100 micrograms/day to 750 micrograms/day, including from 200 micrograms/day to 500 micrograms/day, including from 250 micrograms/day to 350 micrograms/day, and also including about 300 micrograms/day. According to the methods and uses provided herein, in certain embodiments, the nutritional compositions comprise lutein in a total amount of from 1 microgram/day to 1200 micrograms/day, including from 50 micrograms/day to 1000 micrograms/day, including from 100 micrograms/day to 750 micrograms/day, including from 200 micrograms/day to 500 micrograms/day, including from 250 micrograms/day to 350 micrograms/day, and also including about 300 micrograms/day.

Each of the carotenoids in the selected combinations can be obtained from any known or otherwise suitable material source for use in infant formulas, and each can be provided individually, or all together, or in any combination and from any number of sources, including sources such as multivitamin premixes containing other vitamins or minerals in combination with one or more of the carotenoids as described herein. Non-limiting examples of some suitable sources of lutein, lycopene, beta-carotene, or combinations thereof include LycoVit® lycopene (available from BASF, Mount Olive, N.J.), Lyc-O-Mato® tomato extract in oil, powder, or bead form (available from LycoRed Corp., Orange, N.J.), beta-carotene, lutein, or lycopene (available from DSM Nutritional Products, Parsippany, N.J.), FloraGLOO lutein (available from Kemin Health, Des Moines, Iowa), Xangold® Natural Lutein Esters (available from Cognis, Cincinnati, Ohio), and Lucarotin® beta-carotene (available from BASF, Mount Olive, N.J).

### Macronutrients

The nutritional compositions of the present disclosure may further comprise one or more optional macronutrients in addition to the RRR-alpha tocopherol, LC-PUFA, and optional carotenoid described herein. The optional macronutrients include proteins, lipids (in addition to the LC-PUFA), carbohydrates, and combinations thereof. The nutritional compositions are desirably formulated as dietary products containing all three macronutrients.

Macronutrients suitable for use herein include any protein, lipid (in addition to the LC-PUFA), or carbohydrate or source thereof that is known for or otherwise suitable for use in an oral nutritional composition, provided that the optional macronutrient is safe and effective for oral administration and is otherwise compatible with the other ingredients in the nutritional composition.

The concentration or amount of optional lipid (inclusive of the LC-PUFA), carbohydrate, and protein in the nutritional compositions can vary considerably depending upon the particular product form (e.g., bars or other solid dosage forms, milk or soy-based liquids or other clear beverages, reconstitutable powders, etc.) and the various other formulations and targeted dietary needs. These optional macronutrients are most typically formulated within any of the embodied ranges described in the following tables.

| Nutrient % Total Cal. | Embodiment A | Embodiment B | Embodiment C |
|---|---|---|---|
| Carbohydrate | 0-98 | 2-96 | 10-75 |
| Protein | 0-98 | 2-96 | 5-70 |
| Lipid | 0-98 | 2-96 | 20-85 |
| | Embodiment D | Embodiment E | Embodiment F |
| Carbohydrate | 30-50 | 25-50 | 25-50 |
| Protein | 15-35 | 10-30 | 5-30 |
| Lipid | 35-55 | 1-20 | 2-20 |

Each numerical value is optionally preceded by the term “about.”

Optional carbohydrates suitable for use in the nutritional compositions may be simple, complex, or variations or combinations thereof. Non-limiting examples of suitable carbohydrates include hydrolyzed or modified starch or cornstarch, maltodextrin, isomaltulose, sucromalt, glucose polymers, sucrose, corn syrup, corn syrup solids, rice-derived carbohydrate, glucose, fructose, lactose, high fructose corn syrup, honey, sugar alcohols (e.g., maltitol, erythritol, sorbitol), and combinations thereof.

Optional carbohydrates suitable for use herein also include soluble dietary fiber, non-limiting examples of which include gum Arabic, fructooligosaccharides (FOS), sodium carboxymethyl cellulose, guar gum, citrus pectin, low and high methoxy pectin, oat and barley glucans, carrageenan, psyllium and combinations thereof. Insoluble dietary fiber is also suitable as a carbohydrate source herein, non-limiting examples of which include oat hull fiber, pea hull fiber, soy hull fiber, soy cotyledon fiber, sugar beet fiber, cellulose, corn bran, and combinations thereof.

### Protein

Optional proteins suitable for use in the nutritional compositions include hydrolyzed, partially hydrolyzed or non-hydrolyzed proteins or protein sources, and can be derived from any known or otherwise suitable source such as milk (e.g., casein, whey), animal (e.g., meat, fish, egg albumen), cereal (e.g., rice, corn), vegetable (e.g., soy, pea, potato), or combinations thereof. The proteins for use herein can also include, or be entirely or partially replaced by, free amino acids known for use in nutritional products, non-limiting examples of which include L-tryptophan, L-glutamine, L-tyrosine, L-methionine, L-cysteine, taurine, L-arginine, L-carnitine, and combinations thereof.

### Lipid

Optional lipids suitable for use in the nutritional compositions, which are optionally in addition to the LC-PUFAs as described herein, include coconut oil, fractionated coconut oil, soy oil, corn oil, olive oil, safflower oil, high oleic safflower oil, high GLA-safflower oil, MCT oil (medium chain triglycerides), sunflower oil, high oleic sunflower oil, palm and palm kernel oils, palm olein, canola oil, flaxseed oil, borage oil, cottonseed oils, evening primrose oil, blackcurrant seed oil, transgenic oil sources, fungal oils, marine oils (e.g., tuna, sardine), and so forth.

### Optional Ingredients

The nutritional compositions may further comprise other optional ingredients that may modify the physical, nutritional, chemical, hedonic or processing characteristics of the formulas or serve as pharmaceutical or additional nutritional components when used in a targeted population. Many such optional ingredients are known or otherwise suitable for use in other nutritional products and may also be used in the nutritional compositions described herein, provided that such optional ingredients are safe and effective for oral administration and are compatible with the essential and other ingredients in the composition.

Non-limiting examples of such other optional ingredients include preservatives, anti-oxidants, buffers, pharmaceutical actives, sweeteners, colorants, flavors, flavor enhancers, thickening agents and stabilizers, emulsifying agents, lubricants, and combinations thereof.

The nutritional compositions may further include one or more minerals, non-limiting examples of which include phosphorus, sodium, chloride, magnesium, manganese, iron, copper, zinc, iodine, calcium, potassium, chromium, molybdenum, selenium, and combinations thereof.

The nutritional compositions may also include one or more vitamins including vitamin C, non-limiting examples of which include biotin, choline, inositol, folic acid, pantothenic acid, TPAN, choline, vitamin A, thiamine (vitamin B1), riboflavin (vitamin B2) niacin (vitamin B3), pyridoxine (vitamin B6), cyanocobalamin (vitamin B12)—vitamin D, vitamin E, vitamin K, and various salts, esters, or other derivatives thereof, and combinations thereof.

### Methods of Manufacture

The nutritional compositions may be prepared by any known or otherwise effective manufacturing technique for preparing the selected product form. Many such techniques are known for any given product form such as nutritional liquids and nutritional powders and can easily be applied by one of ordinary skill in the nutrition and formulation arts to the nutritional products described herein.

Liquid, milk or soy-based nutritional liquids, for example, may be prepared by first forming an oil and fiber blend containing all formulation oils, any emulsifier, fiber and fat-soluble vitamins. Additional slurries (typically a carbohydrate and two protein slurries) are prepared separately by mixing the carbohydrate and minerals together and the protein in water. The slurries are then mixed together with the oil blend. The resulting mixture is homogenized, heat processed, standardized with any water-soluble vitamins, flavored and the liquid terminally sterilized or aseptically filled or dried to produce a powder.

The nutritional compositions of the present disclosure may also be manufactured by other known or otherwise suitable techniques not specifically described herein without departing from the spirit and scope of the present disclosure. The present embodiments are, therefore, to be considered in all respects as illustrative and not restrictive and that all changes and equivalents also come within the description of the present disclosure.

### Methods of Use

The methods of the present disclosure include the oral administration of the nutritional compositions of this disclosure.

In addition to enhancing brain development, the nutritional compositions can be administered to reduce or prevent the adverse effects of hypoxia on brain and lung development of both fetus and infant, susceptibility of the fetal and newborn brain and lungs to hypoxia and the resulting disorders resulting therefrom including abnormal cognitive development, brain injury and lung injury.

The nutritional compositions as described herein can be administered to individuals including infants as well as pregnant or lactating mothers of such infants or may, in some embodiments, be administered to a specific subclass of infants that are “in need thereof;” that is, to specific infants (or pregnant or lactating mothers of such infants) that would specifically benefit by administration of the infant formula. For example, a specific infant may be “in need of” the infant formulas as described herein if they are susceptible to (i.e., genetically predisposed, have a family history of, and/or having symptoms of the disease or condition) of the disorders resulting from hypoxia including abnormal cognitive development, brain injury and lung injury, mental retardation, seizure disorders, hypoxic-ischemic encephalopathy, cerebral palsy and brain disorder.

The individual desirably consumes at least one serving of the nutritional composition daily, and in some embodiments, may consume two, three, or even more servings per day. Each serving is desirably administered as a single, undivided dose, although the serving may also be divided into two or more partial or divided servings to be taken at two or more times during the day. The methods of the present disclosure include continuous day after day administration, as well as periodic or limited administration, although continuous day after day administration is generally desirable. The methods of the present disclosure are preferably applied on a daily basis, wherein the daily administration is maintained continuously for at least 3 days, including at least 5 days, including at least 1 month, including at least 6 weeks, including at least 8 weeks, including at least 2 months, including at least 6 months, desirably for at least about 18-24 months, desirably as a long term, continuous, daily, dietary source or supplement.

As discussed above, the nutritional composition disclosed herein may have varying concentrations of RRR-alpha tocopherol, LC-PUFA, and optional carotenoid. In certain embodiments of the methods and uses disclosed herein, the nutritional composition contains a sufficient amount of these ingredients (and is consumed in sufficient quantity) to provide the subject consuming the nutritional composition with 300 micrograms to 12,000 micrograms of carotenoid (e.g., lutein) per day. In certain embodiments of the methods and uses disclosed herein, the nutritional composition contains a sufficient amount of these ingredients (and is consumed in sufficient quantity) to provide the subject consuming the nutritional composition with 3 mg to 500 mg of RRR-alpha-tocopherol per day. In certain embodiments of the methods and uses disclosed herein, the nutritional composition contains a sufficient amount of these ingredients (and is consumed in sufficient quantity) to provide the subject consuming the nutritional composition with 5 milligrams to 1200 milligrams of LC-PUFA per day. In certain embodiments of the methods and uses disclosed herein, the nutritional composition contains a sufficient amount of carotenoid (e.g., lutein) and RRR-alpha tocopherol, and is consumed in sufficient quantity, to provide the subject consuming the nutritional composition with 300 micrograms to 12,000 micrograms of carotenoid (e.g., lutein) per day and 3 mg to 500 mg of RRR-alpha tocopherol per day.

Further embodiments include a nutritional composition for use in preventing or reducing susceptibility to a neurological or lung disorder in an infant, child or toddler wherein the composition comprises RRR-alpha tocopherol and LC-PUFA. In certain embodiments, the LC-PUFA is at least one of docosahexaenoic acid, arachidonic acid, docosapentaenoic acid, and gamma linolenic acid. In certain embodiments, the neurological disorder comprises hypoxic injury, an abnormal cognitive development, mental retardation, seizure disorders, hypoxic-ischemic encephalopathy, cerebral palsy or a brain disorder. In certain embodiments, the lung disorder comprises a lung injury that may result in hypoxia. In certain embodiments, the use comprises consumption of the nutritional composition by an infant, a toddler, a child, a pregnant woman, or a lactating woman. In certain embodiments, the infant is a pre-term infant. In certain embodiments, the nutritional composition is in a ready-to-drink liquid form, concentrated form, or as a nutritional liquid made by reconstituting a nutritional powder. In certain embodiments, the composition comprises at least 3 mg/L of RRR-alpha tocopherol. In certain embodiments, the composition comprises at least 20 mg/L of LC-PUFA, including at least 60 mg/L of LC-PUFA. In certain embodiments, the use comprises consumption of the nutritional composition by an infant, a toddler, a child, a pregnant woman, or a lactating woman, in an amount sufficient to provide at least 3 mg of RRR-alpha tocopherol per day of consumption. In certain embodiments, the use comprises consumption of the nutritional composition by an infant, a toddler, a child, a pregnant woman, or a lactating woman, in an amount sufficient to provide at least 20 mg LC-PUFA per day of consumption, preferably at least 60 mg of LC-PUFA per day of consumption. In certain embodiments, the composition comprises at least 20 mg/L of docosahexaenoic acid, including at least 60 mg/L of docosahexaenoic acid, and including from about 72 mg/L to about 144 mg/L of docosahexaenoic acid. In certain embodiments, the use comprises consumption of the nutritional composition by an infant, a toddler, a child, a pregnant woman, or a lactating woman, in an amount sufficient to provide at least 20 mg of docosahexaenoic acid per day of consumption, preferably at least 60 mg of docosahexaenoic acid per day of consumption. In certain embodiments the docosahexaenoic acid is provided in encapsulated or slow release form. In certain embodiments, the composition further comprises from about 50 to about 1150 micrograms/liter of carotenoid. In certain embodiments where the composition includes a carotenoid, the composition has a ratio of RRR-alpha tocopherol (milligrams):carotenoid (micrograms) of 2:1 to 200:1, preferably 10:1 to 100:1. In certain embodiments where the composition includes a carotenoid, the carotenoid is lutein, zeaxanthin, or a combination thereof. In certain embodiments where the composition includes a carotenoid, the use comprises consumption of the nutritional composition by an infant, a toddler, a child, a pregnant woman, or a lactating woman in an amount sufficient to provide 300 micrograms to 12,000 micrograms of carotenoid per day of consumption. In certain embodiments where the composition includes a carotenoid, the use comprises consumption of the nutritional composition by an infant, a toddler, a child, a pregnant woman, or a lactating woman in an amount sufficient to provide 300 micrograms to 12,000 micrograms of lutein per day of consumption. In certain embodiments, the use comprises consumption of the nutritional composition by an infant, a toddler, a child, a pregnant woman, or a lactating woman in an amount sufficient to provide 3 milligrams to 500 milligrams of RRR-alpha tocopherol per day of consumption. In certain embodiments, the use comprises consumption of the nutritional composition by an infant, a toddler, a child, a pregnant woman, or a lactating woman in an amount sufficient to provide 5 milligrams to 1,200 micrograms of LC-PUFA per day of consumption. In certain embodiments, the infant was low birth weight at birth.

Further embodiments include the use of RRR-alpha tocopherol, and a LC-PUFA in the manufacture of a nutritional composition for preventing or reducing susceptibility to a neurological or lung disorder in an infant. In certain embodiments, the neurological disorder comprises hypoxic injury, an abnormal cognitive development, mental retardation, seizure disorders, hypoxic-ischemic encephalopathy, cerebral palsy or a brain disorder.

Further embodiments include a method of preventing or reducing susceptibility to a neurological or lung disorder in a subject in need thereof, the method comprising administration of a LC-PUFA-containing nutritional composition comprising an effective amount of RRR-alpha tocopherol. In certain embodiments, the LC-PUFA is at least one of docosahexaenoic acid, arachidonic acid, docosapentaenoic acid, and gamma linolenic acid. In certain embodiments, the subject in need thereof is an infant. In certain embodiments, the neurological disorder comprises at least one of hypoxic injury, an abnormal cognitive development, mental retardation, seizure disorders, hypoxic-ischemic encephalopathy, cerebral palsy, or a brain disorder. In certain embodiments, the lung disorder comprises a lung injury known to be associated with hypoxia. In certain embodiments, the subject in need thereof is an infant, a toddler, a child, a pregnant woman, or a lactating woman. In certain embodiments, the subject is a pre-term infant. In certain embodiments, the effective amount of RRR-alpha tocopherol comprises at least 3 mg/L. In certain embodiments, the nutritional composition comprises at least 20 mg/L of LC-PUFA, including at least 60 mg/L of LC-PUFA. In certain embodiments, the nutritional composition comprises at least 20 mg/L of docosahexaenoic acid, including at least 60 mg/L of docosahexaenoic acid, and also including from about 72 mg/L to about 144 mg/L of docosahexaenoic acid. In certain embodiments, the docosahexaenoic acid is provided in encapsulated or slow release form. In certain embodiments, the composition further comprises an effective amount of at least one carotenoid. In certain embodiments where the composition further comprises an effective amount of at least one carotenoid, the effective amount of at least one carotenoid comprises a total amount of from about 50 to about 1150 micrograms/liter of carotenoid. In certain embodiments where the composition further comprises an effective amount of at least one carotenoid, the nutritional composition has a ratio of RRR-alpha tocopherol (milligrams):carotenoid (micrograms) of 2:1 to 200:1, preferably 10:1 to 100:1. In certain embodiments where the composition further comprises an effective amount of at least one carotenoid, the at least one carotenoid comprises lutein, zeaxanthin, or a combination thereof. In certain embodiments, the subject in need thereof is a low birth weight infant. In certain embodiments, at least one of the following is met:
a. the effective amount of RRR-alpha-tocopherol comprises at least 3 mg per day of consumption;
b. the method comprises consumption of a sufficient amount of the nutritional composition to provide the subject with 5-1200 mg/day of LC-PUFA;
c. the nutritional composition comprises an effective amount of at least one carotenoid in an amount of 50-12,000 micrograms per day of consumption.
   In certain embodiments where the composition further comprises an effective amount of at least one carotenoid, the LC-PUFA comprises docosahexaenoic acid and the at least one carotenoid comprises lutein.

Further embodiments include a method of preventing or reducing susceptibility to a neurological or lung disorder in an infant, the method comprising administration of a docosahexaenoic acid-containing nutritional composition comprising an effective amount of RRR-alpha tocopherol.

Further embodiments include a method of preventing or reducing susceptibility to a neurological or lung disorder in an infant, the method comprising administration of a docosahexaenoic acid-containing nutritional composition comprising an effective amount of RRR-alpha tocopherol, wherein the neurological disorder comprises at least one of hypoxic injury, an abnormal cognitive development, mental retardation, seizure disorders, hypoxic-ischemic encephalopathy, cerebral palsy, or a brain disorder.

Further embodiments include a method of preventing or reducing susceptibility to a neurological or lung disorder in a subject in need thereof, the method comprising administration of a LC-PUFA-containing nutritional composition comprising an effective amount of RRR-alpha tocopherol and at least one carotenoid. In certain embodiments, the LC-PUFA comprises at least one of: docosahexaenoic acid, arachidonic acid, docosapentaenoic acid, and gamma linolenic acid. In certain embodiments, the at least one carotenoid comprises lutein, zeaxanthin, or a combination thereof. In certain embodiments, the subject in need thereof is an infant. In certain embodiments, the neurological disorder comprises at least one of: hypoxic injury, an abnormal cognitive development, mental retardation, seizure disorders, hypoxic-ischemic encephalopathy, cerebral palsy, or a brain disorder. In certain embodiments, the lung disorder comprises a lung injury known to be associated with hypoxia. In certain embodiments, the subject in need thereof is an infant, a toddler, a child, a pregnant woman, or a lactating woman. In certain embodiments, the subject is a pre-term infant. In certain embodiments, the effective amount of RRR-alpha tocopherol comprises at least 3 mg/L. In certain embodiments, the nutritional composition comprises at least 20 mg/L of LC-PUFA, including at least 60 mg/L of LC-PUFA. In certain embodiments, the nutritional composition comprises at least 20 mg/L of docosahexaenoic acid, including at least 60 mg/L of docosahexaenoic acid, and also including from about 72 mg/L to about 144 mg/L of docosahexaenoic acid. In certain embodiments, the docosahexaenoic acid is provided in encapsulated or slow release form. In certain embodiments, the effective amount of the at least one carotenoid comprises a total amount of from about 50 to about 1150 micrograms/liter of carotenoid. In certain embodiments, the nutritional composition has a ratio of RRR-alpha tocopherol (milligrams):carotenoid (micrograms) of 2:1 to 200:1, preferably 10:1 to 100:1. In certain embodiments, the subject in need thereof is a low birth weight infant.

In certain embodiments, at least one of the following is met:
a. the effective amount of RRR-alpha-tocopherol is at least 3 mg per day of administration;
b. the method comprises consumption of a sufficient amount of the nutritional composition to provide the subject with 5-1200 mg/day of LC-PUFA;
c. the effective amount of at least one carotenoid is 50-12,000 micrograms per day of administration.
   In certain embodiments, the LC-PUFA comprises DHA and the at least one carotenoid comprises lutein.

Further embodiments include a method of preventing or reducing susceptibility to a neurological or lung disorder in an infant, the method comprising administration of a DHA-containing nutritional composition comprising effective amounts of RRR-alpha tocopherol and at least one carotenoid.

Further embodiments include a method of preventing or reducing susceptibility to a neurological or lung disorder in an infant, the method comprising administration of a DHA-containing nutritional composition comprising effective amounts of RRR-alpha tocopherol and at least one carotenoid, wherein the neurological disorder comprises at least one of hypoxic injury, an abnormal cognitive development, mental retardation, seizure disorders, hypoxic-ischemic encephalopathy, cerebral palsy, or a brain disorder.

### EXAMPLES

The following example illustrates specific embodiments and or features of the nutritional products and methods of the present disclosure. The example is given solely for the purpose of illustration and are not to be construed as limitations of the technology described in this disclosure in any way.

### Example 1

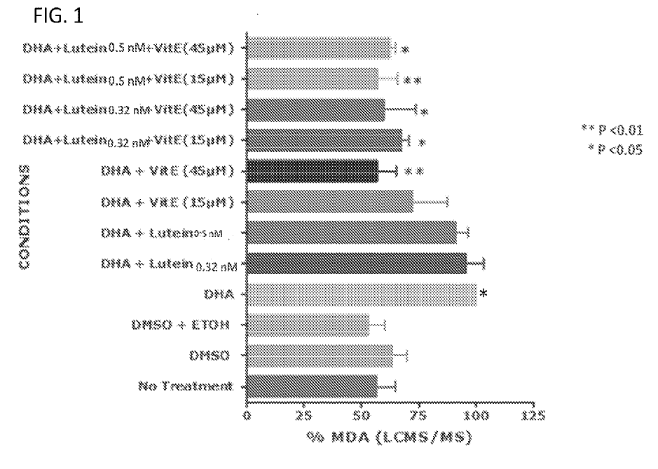

Primary cortical neurons from postnatal day 3-5 (P3-P5) Sprague-Dawley rat pup's brain were pretreated with/without natural vitamin E in the form of RRR-a-tocopherol (15 μM or 45 μM), and/or lutein (0.32 nM or 0.5 nM) individually and in combination for 12 hours. (Note that μM is used to refer to a micromolar concentration and nM is used to refer to a nanomolar concentration and concentrations refer to those reached in the test tubes containing the cortical neurons.) After 12 hours DHA (90 μM) was added for an additional 12 hours. After 12 hours samples were washed in PS and neurons were lysed. Peroxides generated from DHA and incorporated into the neuronal cell membrane were assessed by measuring the MDA levels using liquid chromatography-mass spectometry (LC-MS) method. The results obtained are set forth in the following FIG. 1.

As shown in FIG. 1, without either RRR alpha tocopherol or lutein, DHA undergoes significant peroxidation as reflected by the concentration of MDA obtained in the neurocell membranes of the sacrificed subjects, about 110 LCMS/MS. However, when 45 μM of natural Vitamin E (RRR alpha tocopherol) was included, the resistance against peroxidation was excellent, as reflected by an MDA concentration of only about 57% (p<0.001) by LCMS/MS. On the other hand, when the concentration of RRR alpha tocopherol was reduced to only 15 μM, the resistance against peroxidation was only fair, as reflected by an MDA concentration of only about 75% by LCMS/MS.

However, when 0.5 nM lutein was included in this system, i.e., when both 15 μM RRR alpha tocopherol and 0.5 nM lutein were included, essentially the same excellent resistance against peroxidation was achieved as in the case of 45 μM of RRR alpha tocopherol, as reflected by an MDA concentration also of only about 55% (p<0.001) by LCMS/MS. Thus, including only a small amount of lutein in the system enables the amount of RRR alpha tocopherol needed to achieve excellent resistance against peroxidation to by reduced by 2/3. It was surprising and unexpected that such a small amount of lutein in combination with a relatively lower amount (i.e., about 2/3 less) of RRR alpha tocopherol was able to produce results commensurate with the use of much higher amounts of RRR alpha tocopherol.

It was also noted that the same beneficial effect of adding lutein to RRR alpha tocopherol did not occur when the two were utilized together but the concentration of RRR alpha tocopherol was 45 μM. Also, the use of lutein alone, i.e., without the RRR alpha tocopherol, had essentially no effect on retarding DHA peroxidation.

1. A method for preventing or reducing susceptibility to a neurological or lung disorder in an infant, child or toddler the comprising administering a nutritional composition comprising RRR-alpha tocopherol, a long chain polyunsaturated fatty acid (LC-PUFA), and a carotenoid to the infant, child, or toddler, or to a pregnant or lactating woman.

2. The method according to claim 1, wherein the LC-PUFA is selected from the group consisting of docosahexaenoic acid, arachidonic acid, docosapentaenoic acid, gamma linolenic acid, and combinations thereof.

3. (canceled)

4. The method according to claim 1, wherein the carotenoid is selected from the group consisting of lutein, zeaxanthin, beta carotene, lycopene, and combinations thereof.

5. The method according to claim 1, wherein the composition has a ratio of RRR-alpha tocopherol (milligrams):carotenoid (micrograms) of 2:1 to 200:1, preferably 10:1 to 100:1.

6. The method according to claim 1, wherein the neurological disorder comprises hypoxic injury, an abnormal cognitive development, mental retardation, seizure disorders, hypoxic-ischemic encephalopathy, cerebral palsy or a brain disorder.

7. The method according to claim 1, wherein the lung disorder comprises a lung injury that may result in hypoxia.

8. (canceled)

9. The method according to claim 1 for use in pre-term infants.

10. The method according to claim 1, wherein the nutritional composition is in ready-to-drink liquid form, concentrated form, or is a nutritional liquid made by reconstituting a nutritional powder.

11. The method according to claim 1, wherein the nutritional composition comprises at least 3 mg/L of RRR-alpha tocopherol.

12. The method according to claim 1, wherein the composition comprises at least 20 mg/L of LC-PUFA.

13. (canceled)

14. The method according to claim 1, wherein the nutritional composition is administered to an infant, a toddler, a child, a pregnant woman, or a lactating woman in an amount sufficient to provide at least 3 mg of RRR-alpha tocopherol per day of consumption.

15. The method according to claim 1, wherein the nutritional composition is administered to an infant, a toddler, a child, a pregnant woman, or a lactating woman, in an amount sufficient to provide at least 20 mg LC-PUFA per day of consumption.

16. The method according to claim 2, wherein the nutritional composition comprises at least 20 mg/L of docosahexaenoic acid.

17. (canceled)

18. (canceled)

19. The method according to claim 16, wherein the nutritional composition is administered to an infant, a toddler, a child, a pregnant woman, or a lactating woman in an amount sufficient to provide at least 20 mg of docosahexaenoic acid per day of consumption.

20. The method according to claim 16, wherein the docosahexaenoic acid is provided in encapsulated or slow release form.

21. The method according to claim 1, wherein the composition comprises from about 50 to about 1150 micrograms/liter of carotenoid.

22. The method according to claim 1, wherein the nutritional composition is administered to an infant, a toddler, a child, a pregnant woman, or a lactating woman in an amount sufficient to provide 300 micrograms to 12,000 micrograms of carotenoid per day of consumption.

23. The method according to claim 22, wherein the carotenoid comprises lutein.

24. (canceled)

25. The method according to claim 1, wherein the nutritional composition is administered to an infant, a toddler, a child, a pregnant woman, or a lactating woman in an amount sufficient to provide 5 milligrams to 1,200 micrograms of LC-PUFA per day of consumption.

26. The method according to claim 1, wherein the infant was low birth weight at birth.

27. (canceled)

28. (canceled)
